(19) United States
(12) Patent Application Publication
(10) Pub. No.: US 2020/0214667 A1
(43) Pub. Date: Jul. 9, 2020

(54) ULTRASOUND PROBE NAVIGATION USING A HAPTIC FEEDBACK DEVICE

(71) Applicant: General Electric Company, Schenectady, NY (US)

(72) Inventors: David Andrew Shoudy, Niskayuna, NY (US); Ratnadeep Paul, Cohoes, NY (US); Bryan Hayes, Clinton, MA (US)

(21) Appl. No.: 16/241,785

(22) Filed: Jan. 7, 2019

(51) Int. Cl. A61B 8/00; A61B 8/14

(52) U.S. Cl. CPC A61B 8/4254; G06F 3/016; A61B 8/4444; A61B 8/14

(57) ABSTRACT

A wearable device for providing ultrasound guidance feedback may include a housing that has a plurality of haptic actuators, and an opening extending along an axis through the housing. The opening may fit about a body part of an operator of an ultrasound probe, and the haptic actuators may pulse in an actuation pattern indicative of one or more suggested movements of the ultrasound probe to position the ultrasound probe in a desired position to acquire imaging data of a region of interest of a patient.

### BACKGROUND

The subject matter disclosed herein relates to medical imaging, and more particularly to systems and methods for guiding ultrasound probe positioning through haptic feedback.

An ultrasound imaging system typically includes an ultrasound probe that is applied to a patient's body and a workstation or monitor that is operably coupled to the ultrasound probe. The ultrasound probe may be controlled by an operator of the ultrasound imaging system and is configured to transmit and receive ultrasound signals that are processed into an ultrasound image by the workstation or monitor. The operator positions the ultrasound probe to acquire a target anatomy or region of interest (e.g., a desired tissue or body region to be imaged) in a target scan plane. For example, by viewing real-time images of the acquired ultrasound data on the monitor or a separate display of the ultrasound imaging system, the operator may adjust the ultrasound probe into an appropriate position for imaging the target scan plane of the target region of interest. However, it is recognized that there may be some challenges with such positioning methods. For example, manually finding the appropriate position of the ultrasound probe via viewing the displayed images alone may be difficult, time consuming, and result in less accurate positioning, especially for unskilled users.

### BRIEF DESCRIPTION

A summary of certain embodiments disclosed herein is set forth below. It should be understood that these aspects are presented merely to provide the reader with a brief summary of these certain embodiments and that these aspects are not intended to limit the scope of this disclosure. Indeed, this disclosure may encompass a variety of aspects that may not be set forth below.

In one embodiment, an ultrasound imaging system includes an ultrasound probe. The ultrasound imaging system further includes a monitor having a memory and a processor. The processor is communicatively coupled to the ultrasound probe and a haptic feedback device having a plurality of haptic actuators. The processor may determine a current position of the ultrasound probe relative to a region of interest of a patient. The region of interest may include a desired anatomy, a desired scan plane, or both, to be imaged via the ultrasound probe. The processor may also determine whether the current position of the ultrasound probe corresponds to a desired position of the ultrasound probe relative to the region of interest. The desired position of the ultrasound probe facilitates an acquisition of an ultrasound image of the region of interest. In response to determining that the current position of the ultrasound probe does not correspond to the desired position of the ultrasound probe, the processor may transmit a control signal to a plurality of haptic actuators. The control signal may cause the plurality of actuators to pulse in a actuation pattern indicative of a suggested movement of the ultrasound probe to position the ultrasound probe in the desired position.

In another embodiment, a wearable device for providing ultrasound guidance feedback may include a housing that has a plurality of haptic actuators, and an opening extending along an axis through the housing. The opening may fit about a body part of an operator of an ultrasound probe, and the haptic actuators may pulse in an actuation pattern indicative of one or more suggested movements of the ultrasound probe to position the ultrasound probe in a desired position to acquire imaging data of a region of interest of a patient.

In another embodiment, a method may include determining, via a processor, a desired imaging position of the ultrasound probe based at least in part on the region of interest and the desired scan plane. The desired imaging position may facilitate an acquisition of an ultrasound image of the region of interest, the desired scan plane, or both. The method may also include determining, via the processor, a current position of the ultrasound probe relative to the target region of interest, the desired scan plane, or both, determining, via the processor, whether the current position of the ultrasound probe corresponds to the desired imaging position of the ultrasound probe, and in response to determining that the current position of the ultrasound probe does not correspond to the desired imaging position of the ultrasound probe, transmitting, via the processor, a control signal to a plurality of haptic actuators disposed about a haptic feedback device configured to be worn by an operator of the ultrasound probe. The control signal may cause the plurality of haptic actuators to pulse in an actuation pattern indicative of a suggested movement of the ultrasound probe to position the ultrasound probe in the desired imaging position.

### BRIEF DESCRIPTION OF THE DRAWINGS

These and other features, aspects, and advantages of the present invention will become better understood when the following detailed description is read with reference to the accompanying drawings in which like characters represent like parts throughout the drawings, wherein:

FIG. 1 illustrates a block diagram of an embodiment of an ultrasound imaging system having a haptic guidance system and a haptic feedback device, in accordance with aspects of the present disclosure;

FIG. 2 illustrates a perspective view of an embodiment of the haptic feedback device of FIG. 1, in accordance with aspects of the present disclosure;

FIG. 3 illustrates a side view of an embodiment of the haptic feedback device of FIG. 1, in accordance with aspects of the present disclosure;

FIG. 4 illustrates an overhead view of an embodiment of the haptic feedback device of FIG. 1, in accordance with aspects of the present disclosure;

FIG. 5 illustrates a perspective view of an embodiment of the haptic feedback device of FIG. 1 worn about a hand of the operator of an ultrasound probe, in accordance with aspects of the present disclosure;

FIG. 6 illustrates a schematic of the haptic feedback device of FIG. 1 showing an embodiment of a vibration sequence of vibration actuators of the haptic feedback device to guide movement of an ultrasound probe along an X-axis, in accordance with aspects of the present disclosure;

FIG. 7 illustrates a schematic of the haptic feedback device of FIG. 1 showing an embodiment of a vibration sequence of the vibration actuators of the haptic feedback device to guide movement of an ultrasound probe along a Y-axis, in accordance with aspects of the present disclosure;

FIG. 8 illustrates a schematic of the haptic feedback device of FIG. 1 showing an embodiment of a vibration sequence of the vibration actuators of the haptic feedback device to guide compression movement of an ultrasound probe along a Z-axis, in accordance with aspects of the present disclosure;

FIG. 9 illustrates a schematic of the haptic feedback device of FIG. 1 showing an embodiment of a vibration sequence of the vibration actuators of the haptic feedback device to guide tilting movement of the ultrasound probe along an elevation direction, in accordance with aspects of the present disclosure;

FIG. 10 illustrates a schematic of the haptic feedback device of FIG. 1 showing an embodiment of a vibration sequence of the vibration actuators of the haptic feedback device to guide rocking movement of the ultrasound probe along an azimuth direction, in accordance with aspects of the present disclosure;

FIG. 11 illustrates a schematic of the haptic feedback device of FIG. 1 showing an embodiment of a vibration sequence of the vibration actuators of the haptic feedback device to guide rotational movement of the ultrasound probe, in accordance with aspects of the present disclosure; and

FIG. 12 illustrates a flow diagram of an embodiment of a method for guiding movement of the ultrasound probe using the haptic guidance system of FIG. 1, in accordance with aspects of the present disclosure.

### DETAILED DESCRIPTION

One or more specific embodiments will be described below. In an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments of the present disclosure, the articles “a,” “an,” “the,” and “said” are intended to mean that there are one or more of the elements. The terms “comprising,” “including,” and “having” are intended to be inclusive and mean that there may be additional elements other than the listed elements. Furthermore, any numerical examples in the following discussion are intended to be non-limiting, and thus additional numerical values, ranges, and percentages are within the scope of the disclosed embodiments.

As mentioned above, ultrasound imaging systems typically include an ultrasound probe that is applied to a patient's body. Ultrasound imaging systems may be operated by skilled technicians trained in locating the target region of interest on the patient to be imaged and the target scan plan, generally by using the ultrasound image data as guidance. A challenge in the field of ultrasound to widespread growth and adoption of ultrasound imaging systems in every physician's office and throughout the developing world is the lack of skilled sonographers (e.g., technicians) or operators capable of performing such interpretation of the data and manipulation of the probe. For example, to accurately perform ultrasound imaging of a desired anatomy of a patient, an operator must properly align an ultrasound probe of an ultrasound system with the desired anatomy, interpret ultrasound images while controlling settings of the ultrasound system to bring the desired anatomy into view, and navigate the ultrasound probe to identify the appropriate scan planes to store for the examination of the patient. As such, it may be desirable for the ultrasound imaging system to provide guidance to less skilled operators so that such operators may obtain proper ultrasound images for diagnosis. Accordingly, an operator-independent ultrasound imaging system may automate some operational steps that are typically performed by an operator and provide machine guidance to the operator to perform other operational steps. For example, the operator-independent ultrasound imaging system may automate many selections of parameters and/or characteristics that an operator would conventionally adjust to perform ultrasound imaging of a patient. Additionally, a haptic feedback device is provided herein to provide guided ultrasound probe placement to the operator to acquire a desired scan plane of a desired anatomical feature of a patient. For example, the haptic feedback device may assist an operator in positioning and orienting an ultrasound probe to acquire the desired scan plane of a desired anatomical feature of a patient. Such techniques may allow novice, or less skilled, users to obtain accurate ultrasound scans of desired anatomical features of a patient.

For example, a haptic feedback device (e.g., band or glove) may be worn by an operator and provide vibrational feedback (e.g., haptic feedback) to the operator to assist in positioning and orienting an ultrasound probe to acquire a desired scan plane of a desired anatomical feature of a patient. In one embodiment, the haptic feedback device and the ultrasound probe may each have one or more position and/or orientation sensors, such as inertial measurement units. An operator-independent ultrasound imaging system in communication with the haptic feedback device and the ultrasound imaging system may receive the positional and/or orientation data from the haptic feedback device, the ultrasound probe, or both. The ultrasound imaging system may compare the position and/or orientation of the ultrasound probe with a patient-centric coordinate system (e.g., an anatomical atlas) of a patient. Based on the comparison of the position and/or orientation of the ultrasound probe with the patient-centric coordinate system, the ultrasound imaging system may determine a directional movement, an angular movement, or both, to assist the operator in positioning and/or orienting the ultrasound probe to acquire a desired scan plane of desired anatomical features of the patient. The ultrasound imaging system may then determine the relative orientation of haptic feedback device with respect to the ultrasound probe based on a comparison between the ultrasound probe orientation data and the haptic feedback device orientation data. Based on the relative orientation between the ultrasound probe and haptic feedback device and/or the determined directional movement, angular movement, or both, the ultrasound imaging system may then compute and send a command signal to the haptic feedback device to provide to the operator vibrational feedback that corresponds to the determined directional movement, angular movement, or both. In response to sensing the vibrational feedback through the haptic feedback device, the operator may discern a direction, a change in direction, an orientation, a change in orientation, or the like, that corresponds to the particular vibrational feedback received by the operator. For example, the vibrational feedback may correspond to a command to move the ultrasound probe to the right, to the left, forward, backward, to apply pressure at the current location, to apply less pressure at the current location, to tilt to the right, to tilt to the left, to tilt forward, to tilt backward, to rotate clockwise, to rotate counter-clockwise, or the like. The operator may then move the ultrasound probe in the corresponding direction and/or angle to acquire the desired scan plane of the desired anatomical features of the patient.

In some embodiments, the ultrasound imaging system may provide vibrational feedback to the operator through the haptic feedback device more than once before the operator is able to acquire the desired scan plane of the desired anatomical features of the patient. For example, the ultrasound imaging system may direct the operator to move the ultrasound probe to the right and then rotate the ultrasound probe clockwise to acquire the desired scan plane of the desired anatomical features of the patient. It should be noted that the ultrasound imaging system may direct the operator to move (or not move) the ultrasound probe in any combination of directions and/or angles to assist the operator in positioning and/or orienting the ultrasound probe to acquire the desired scan plane of the desired anatomical features of the patient.

In another embodiment, the haptic feedback device may be able to be used with ultrasound probes without inertial measurement units. That is, the haptic feedback device may have one or more inertial measurement units, but the ultrasound probe may not have any inertial measurement units. In such an embodiment, the ultrasound imaging system may receive positional and/or orientation data from the haptic feedback device. The ultrasound imaging system may then compare the position and/or orientation to ultrasound image data acquired via the ultrasound probe. In some embodiments, the ultrasound image data may be acquired in real-time or substantially in real-time. Based on the comparison of the position and/or orientation of the haptic feedback device with the ultrasound image data, the ultrasound imaging system may determine a directional movement, an angular movement, or both, to assist the operator in positioning and/or orienting the ultrasound probe to acquire the desired scan plane of the desired anatomical features of the patient. The ultrasound imaging system may then send a command signal to the haptic feedback device to provide to the operator vibrational feedback that corresponds to the determined directional movement, the angular movement, or both. As such, the haptic feedback device may work with different types of ultrasound probes to provide guided ultrasound probe placement to the operator to acquire the desired scan plane of the desired anatomical feature of the patient.

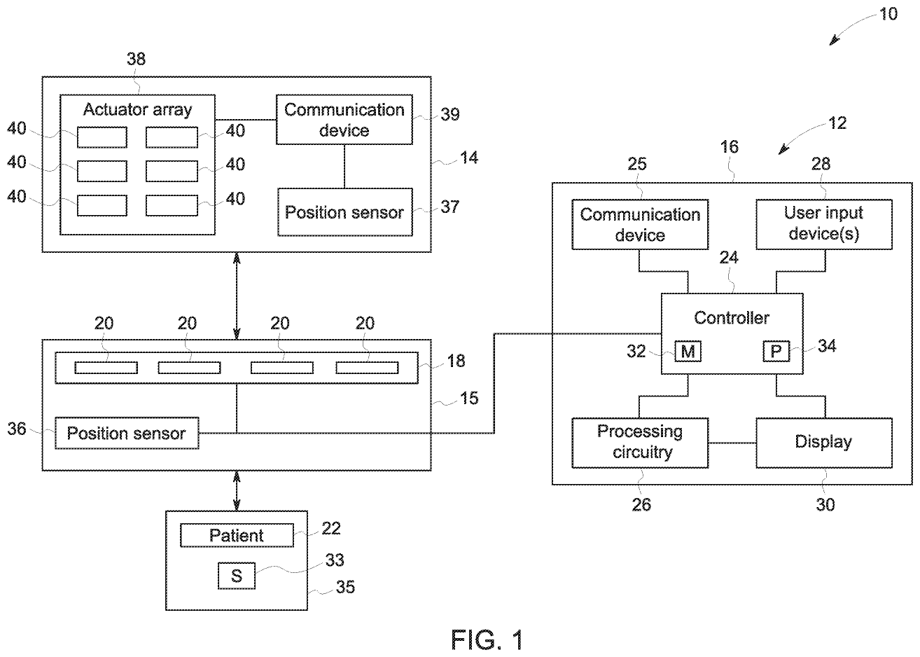

With the foregoing in mind, FIG. 1 illustrates a block diagram of an embodiment of an ultrasound imaging system 10 having a haptic guidance system 12 (e.g., haptic feedback system) that may be used to provide haptic feedback via a haptic feedback device 14 to an operator of the ultrasound imaging system 10 to guide placement of an ultrasound probe 15 of the ultrasound imaging system 10. For example, the haptic feedback device 14 may include a haptic glove, a haptic band, a smartphone, a smartwatch, or any other suitable device that may provide haptic feedback to the operator of the ultrasound imaging system 10.

In the illustrated embodiment, the ultrasound system 10 is a digital acquisition and beam former system, but in other embodiments, the ultrasound system 10 may be any suitable type of ultrasound system, not limited to the illustrated type. The ultrasound system 10 may include the ultrasound probe 15 and a workstation 16 (e.g., monitor, console, user interface) which may control operation of the ultrasound probe 15 and may process image data acquired by the ultrasound probe 15. The ultrasound probe 15 may be coupled to the workstation 16 by any suitable technique for communicating image data and control signals between the ultrasound probe 15 and the workstation 16 such as a wireless, optical, coaxial, or other suitable connection.

The ultrasound probe 15 contacts the patient during an ultrasound examination. The ultrasound probe 15 may include a patient facing or contacting surface that includes a transducer array 18 having a plurality of transducer elements 20. Each individual transducer element 20 may be capable of converting electrical energy into mechanical energy for transmission and mechanical energy into electrical energy for receiving. It should be noted that the transducer array 18 may be configured as a two-way transducer capable of transmitting ultrasound waves into and receiving such energy from a subject or patient 22 during operation when the ultrasound probe 15 is placed in contact with the patient 22. More specifically, the transducer elements 20 may convert electrical energy from the ultrasound probe 15 into ultrasound waves (e.g., ultrasound energy, acoustic waves) and transmit the ultrasound waves into the patient 22. The ultrasound waves may be reflected back toward the transducer array 18, such as from tissue of the patient 22, and the transducer elements 20 may convert the ultrasound energy received from the patient 22 (reflected signals or echoes) into electrical signals for transmission and processing by the ultrasound probe 15 and the workstation 16 to provide an ultrasound image that may be analyzed. The number of transducer elements 20 in the transducer array 18 and the frequencies at which the transducer elements 20 operate may vary depending on the application.

As previously discussed, the ultrasound probe 15 is communicatively coupled to the workstation 16 of the ultrasound imaging system 10 to facilitate image collection and processing. As will be appreciated, the workstation 16 may include a number of elements to control operation of the ultrasound probe 15, facilitate placement guidance of the ultrasound probe 15 via the haptic feedback device 14, and facilitate production of ultrasound images. For instance, as illustrated, the workstation 16 may include a controller 24, processing circuitry 26, one or more user input devices 28, and a display 20. In certain embodiments, the workstation 16 may include additional elements not shown in FIG. 1, such as additional data acquisition and processing controls, additional image display panels, multiple user interfaces, and so forth.

The controller 24 may include a memory 32 and a processor 34. In some embodiments, the memory 32 may include one or more tangible, non-transitory, computer-readable media that store instructions executable by the processor 34 and/or data to be processed by the processor 34. For example, the memory 32 may include random access memory (RAM), read only memory (ROM), rewritable non-volatile memory such as flash memory, hard drives, optical discs, and/or the like. Additionally, the processor 34 may include one or more general purpose microprocessors, one or more application specific processors (ASICs), one or more field programmable logic arrays (FPGAs), or any combination thereof. The controller 24 may control transmission of the ultrasound waves into the patient 22 via the transducer array 18. Additionally, the controller 24 may be part of the haptic guidance system 12 of the ultrasound system 10 and may control other elements of the haptic guidance system 12 to guide placement of the ultrasound probe 15 via the haptic feedback device 14 for acquiring ultrasound images of a desired scan plane of desired anatomical features of a patient 22, as discussed in greater detail below.

The processing circuitry 26 may include receiving and conversion circuitry. The processing circuitry 26 may receive the electrical signal data from the transducer array 18 of the ultrasound probe 15 representing reflected ultrasound energy returned from tissue interfaces within the patient 22. The processing circuitry 26 may process the data from the transducer array 18, such as correcting for noise artifacts, or the like. The processing circuitry 26 may then convert the signal data into an ultrasound image for presentation via the display 30. The controller 24 may cause display of the ultrasound image or images produced by the processing circuitry 26 from the signal data received from the transducer array 18 of the ultrasound probe 15.

The controller 24 may also include a communication device 25 that enables the workstation 16 to communicate data between the workstation 16 and the haptic feedback device 14 and/or the ultrasound probe 15. For example, in the illustrated embodiment, the haptic feedback device 14 may have a communication device 39 that includes a network interface that may enable the haptic feedback device 14 to communicate via various protocols such as various wired or wireless communication protocols, such as Wi-Fi, mobile telecommunications technology (e.g., 2G, 3G, 4G, or LTE), Bluetooth®, near-field-communications technology, and the like.

In operation, the controller 24 may receive a signal (e.g., a user selection, or input) indicative of a desired anatomical feature of the patient 22 and/or a desired scan plane of the desired anatomical feature via the one or more user input devices 28 of the workstation 16. The one or more user input devices 28 may include a keyboard, a touchscreen, a mouse, buttons, switches, or other devices suitable to allow the operator to input the desired anatomical feature and/or the desired scan plane of the desired anatomical feature. Based on the desired anatomical feature and/or the desired scan plane of the desired anatomical feature, the controller 24 may output a signal to the transducer array 18 of the ultrasound probe 15 indicative of an instruction to convert the electrical energy from the ultrasound probe 15 into ultrasound waves and transmit the ultrasound waves into the patient 22 and to detect the ultrasound energy that is reflected back from the tissue interfaces within the patient 22.

The controller 24 may determine that the ultrasound probe 15 is in an appropriate position and/or orientation (e.g., desired position and/or orientation) to sufficiently image the desired anatomical feature in the desired scan plane. In some embodiments, the haptic guidance system 12 of the ultrasound imaging system 10 may include sensing mechanisms to detect the location of the patient and to determine the position and/or orientation of the haptic feedback device 14 and/or the ultrasound probe 15 with respect to the patient 22. That is, the haptic guidance system 12 may include one or more patient position and/or orientation sensors 33, such as weight sensors, contact sensors, cameras, or other suitable sensing mechanisms, disposed about the imaging space 35 of the ultrasound imaging system 10 in which the patient 22 is positioned during ultrasound imaging, or at any other suitable position about the ultrasound imaging system 10 suitable for detecting the position of the patient 22. The patient position and/or orientation sensor 33 may be communicatively coupled to the controller 24 via a wired or wireless connection and may send one or more signals to the controller 24 indicative of the position of the patient about the imaging space 35. Additionally or alternatively, in some embodiments, a position of the patient 22 relative to the imaging space 35 or the ultrasound imaging system 10 may be determined based at least in part on received demographic information, such as height and weight, of the patient 22. For example, such demographic information of the patient 22 may be compared to lookup tables stored in the memory 32 to determine the position of the patient 22 relative to the imaging space 35.

Based at least in part on the one or more signals received from the patient position sensor 33 and/or the received demographic information of the patient 22, the controller may determine, develop, or update a patient-centric coordinate system or map that may be used to track the position of the ultrasound probe 15 relative to the patient 22. For example, a general patient coordinate system based on the imaging space 35 may be stored in the memory 32 and the controller 24 may update the general patient coordinate system based at least in part on the signals received from the patient position sensor 33 and/or the received demographic information of the patient 22 to generate the patient-centric coordinate system. As another example, the controller 24 may develop a new patient-centric coordinate system for each patient based at least in part on the signals received from the patient position sensor 33 and/or the received demographic information of the patient 22. In some embodiments, the patient-centric coordinate system may be created based on an anatomical atlas of the patient. For example, the anatomical atlas may be a voxelated model that represent polygonal 3D structures of internal organs, bony structures, foreign bodies, or the like. The anatomical atlas may be deformable to fit a representation of the patient (e.g., optical camera images).

Additionally or alternatively, in some embodiments, the patient position sensor 33 may include an ultrasound probe with integrated position tracking. In such embodiments, an operator may follow a procedure while using the ultrasound probe with integrated position tracking to place the probe on key landmarks or drag the probe across the surface of the patient until the coordinate system is developed. Additionally or alternatively, in some embodiments, the patient-centric coordinate system may be derived based at least in part on image data received from one or more cameras of the ultrasound imaging system 10 that may be used to measure or determine patient position and/or the live ultrasound image data from the ultrasound probe 15. The patient-centric coordinate system may allow the controller 24 to determine a relative position of the target anatomy within the patient 22 and to determine when the ultrasound probe 15 is positioned appropriately to sufficiently image the target anatomy in the target scan plane. Additionally, the patient-centric coordinate system may be compared or fused with a model of general body anatomy, which may be stored in the memory 32.

The ultrasound probe 15 may include a probe position sensor 36 for detecting the position and/or orientation of the ultrasound probe 15. The probe position sensor 36 may be disposed about the ultrasound probe 15 and may be a position sensor, an orientation sensor, such as a gyroscope, an inertial measurement unit, electromagnetic tracking, optical tracking, or any other suitable sensor that may allow for detection of a current position and/or orientation of the ultrasound probe 15. The probe position sensor 36 may be communicatively coupled to the controller 24 via a wired or wireless connection and may send one or more signals to the controller 24 indicative of a current position and/or orientation of the ultrasound probe 15. Additionally, or alternatively, the haptic feedback device 14 may include a position sensor 37 for detecting the position and/or orientation of the hand of the operator. The position sensor 37 may be disposed about the haptic feedback device 14 and may be a position sensor, an orientation sensor, such as a gyroscope, an inertial measurement unit, electromagnetic tracking, optical tracking, or any other suitable sensor that may allow for detection of a current position and/or orientation of the hand of the operator. The position sensor 37 may be communicatively coupled to the controller 24 via a wired or wireless connection (e.g., via communication device 39) and may send one or more signals to the controller 24 indicative of a current position and/or orientation of the hand or wrist of the operator.

The controller 24 may compare the current position and/or orientation of the ultrasound probe 15 and/or the haptic feedback device 14, based at least in part on respective signals received from the probe position sensor 36 and position sensor 37 of the haptic feedback device 14, to the patient-centric coordinate system developed based at least in part on the signals received from the patient position sensor 33 and/or the live ultrasound image data. Based on the comparison, the controller 24 may determine whether the ultrasound probe 15 and/or the haptic feedback device 14 (i.e., the operator's hand) is in the appropriate position and/or orientation to sufficiently image the desired anatomy in the desired scan plane. As such, live ultrasound image data may be used by the controller 24 as feedback to determine if the ultrasound probe 15 and/or the haptic feedback device 14 is in the appropriate position and orientation. The patient-centric coordinate system and the ultrasound image data may be stored in the memory 32.

Additionally, or alternatively, the controller 24 may compare the current position and/or orientation of the ultrasound probe 15, based at least in part on the signals received from the probe position sensor 36, to the position and/or orientation of the haptic feedback device 14 based at least in part on the signals received from the position sensor 37. Based on the comparison, the controller 24 may determine the grip style of the operator's hand on the ultrasound probe 15. For example, the position and/or orientation data of the ultrasound probe 15 may include a plurality of probe reference points (e.g., x-coordinate, y-coordinate, z-coordinate) relative to the imaging space 35, and the position and/or orientation data of the haptic feedback device 14 may include a plurality haptic feedback device reference points (e.g., x-coordinate, y-coordinate, z-coordinate) relative to the imaging space 35. The controller 24 may compare the relative positions of the probe reference points and the haptic feedback device reference points in the imaging space 35 to lookup tables stored in the memory 32 to determine the grip style of the operator's hand on the ultrasound probe 15. As such, the controller 24 may provide haptic feedback to the operator via the haptic feedback device 14 based on the grip style of the operator and the position and/or orientation of the ultrasound probe 15. For example, the controller 24 may adjust a particular vibration pattern of the vibration actuators 40 such that the operator may discern the same vibration pattern from the haptic feedback device 14 regardless of the grip style used by the operator.

In some embodiments, the controller 24 may control the transducer array 18 and the processing circuitry 26 to obtain and generate ultrasound images while the controller 24 is determining whether the ultrasound probe 15 and/or the haptic feedback device 14 is in the appropriate position and/or orientation to sufficiently image the target anatomy in the target scan plane. As such, live ultrasound image data may be used by the controller 24 as feedback in determining if the ultrasound probe 15 and/or the haptic feedback device 14 is in the appropriate position and/or orientation to sufficiently image the target anatomy in the target scan plane. Once the controller 24 determines that the ultrasound probe 15 and/or the haptic feedback device 14 is in the appropriate position and/or orientation to sufficiently image the target anatomy in the target scan plane, the controller 24 may cause the display of a notification, such as a visual or audible notification. Additionally, or alternatively, the controller 24 may send a signal to the vibration actuators 40 of the haptic feedback device 14 indicative of a particular pulse pattern to convey to the operator of the ultrasound probe 15 that the ultrasound probe 15 and/or the haptic feedback device 14 is in the appropriate position and/or orientation to sufficiently image the target anatomy in the target scan plane.

The controller 24 may cause automatic storage in the memory 32 of the live ultrasound image data once it is determined that the ultrasound probe 15 and/or the haptic feedback device 14 is in the appropriate position and/or orientation. Additionally, or alternatively, if the controller 24 determines that the ultrasound probe 15 and/or the haptic feedback device 14 is in the appropriate position and/or orientation, the controller 24 may send a signal to the processing circuitry 26 indicative of an instruction to convert the data received from the transducer array 18 of the ultrasound probe 15 into an ultrasound image and send a signal to the display 30 indicative of an instruction to display the ultrasound image.

In some embodiments, the controller 24 may not cause the transducer array 18 to produce and transmit the ultrasound waves into the patient 22 and detect the reflected ultrasound energy until it is determined that the ultrasound probe 15 and/or the haptic feedback device 14 is in the appropriate position and/or orientation to sufficiently image the desired anatomy in the desired scan plane. If the controller 24 determines that the ultrasound probe 15 and/or the haptic feedback device 14 is not in the appropriate position and/or orientation to sufficiently image the desired anatomy in the desired scan plane, the controller 24 may guide the operator of the ultrasound probe 15 to move the ultrasound probe 15 into the appropriate position via the haptic feedback device 14. As such, the haptic feedback device 14 may include an actuator array 38 that includes multiple vibration actuators 40 (e.g., haptic actuators). In one embodiment, the haptic feedback device 14 may be a band or a glove that fits about a hand of the operator of the ultrasound probe 15. The vibration actuators 40 may be arranged in a particular arrangement around the band to convey to the operator various feedback based on the position and/or orientation of the ultrasound probe 15 with respect to the desired anatomy of the patient and/or the desired scan plane to be imaged. For example, the vibration actuators 40 may be arranged in one row, two rows, three rows, or any other suitable number of rows around the band such that the operator may feel vibrations in various patterns that indicate respective instructions in moving the ultrasound probe, handling the ultrasound probe, or the like. In one embodiment, the instructions may represent a direction of movement associated with the ultrasound probe, a change in orientation of the ultrasound probe, a change in pressure applied to the ultrasound probe, and the like.

In another embodiment, the haptic feedback device 14 may be a smartphone or a smartwatch. The controller 24 may send a signal to the smartphone or the smartwatch to provide haptic feedback to the operator when the controller 24 determines that the ultrasound probe 15 is in the appropriate position to sufficiently image the desired anatomy in the desired scan plane. In some embodiments, the controller 24 may send a signal to the smartphone or the smartwatch to provide haptic feedback in increasing frequency as the ultrasound probe 15 is moved closer to the appropriate position to sufficiently image the desired anatomy in the desired scan plane and provide haptic feedback in decreasing frequency as the ultrasound probe 15 is moved away from the appropriate position to sufficient image the desired anatomy in the desired scan plane.

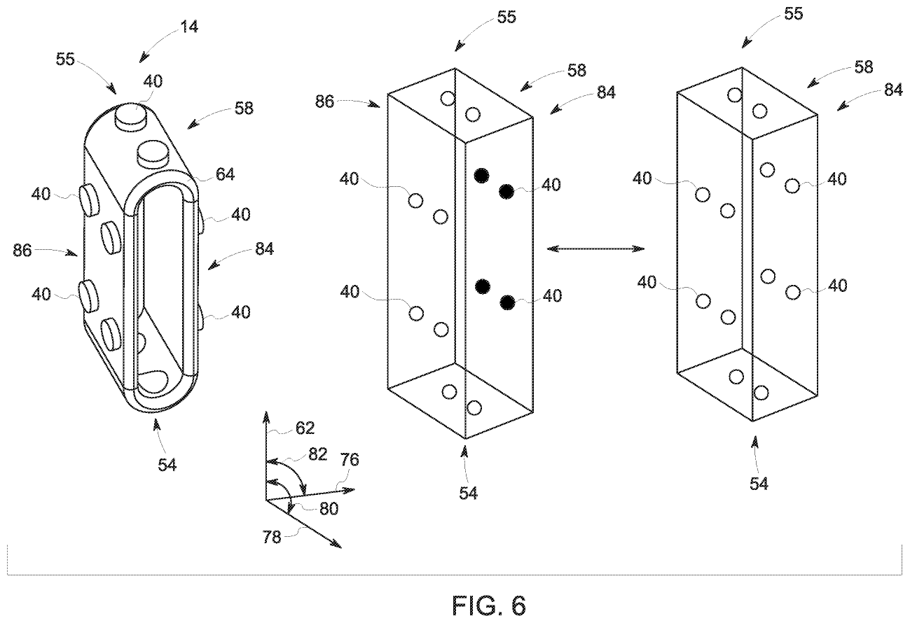

In any case, the controller 24 may send one or more signals to one or more of the vibration actuators 40 to cause the vibration actuators 40 to vibrate in a particular pattern indicative of a particular movement of the ultrasound probe 15 to guide the operator to move the ultrasound probe 15 into the appropriate position and/or orientation. The vibration actuators 40 may be driven by the controller 24 to guide positioning of the ultrasound probe 15 in six degrees-of-freedom (DOF) (i.e., spatial position and angular orientation) by using various vibration patterns. Additional details discussing the vibrational patterns is discussed herein with regard to FIGS. 6-11. By placing the vibration actuators 40 on some or all sides of the haptic feedback device 14, multiple grip styles of the ultrasound probe 15 may be supported. Once the operator has moved the ultrasound probe 15 in the appropriate position to image the desired anatomy, the operator may further be guided by the controller 24 to make fine adjustments to the position and/or orientation of the ultrasound probe 15 to acquire the desired scan plane. As discussed above, in some embodiments, the controller 24 of the ultrasound imaging system 10 may use live (e.g., real-time) ultrasound image data and image processing technology to detect the anatomy and guide this fine positioning while comparing the live ultrasound image data to the desired scan plane.

With the foregoing in mind, the haptic guidance system 12 of the ultrasound imaging system 10 may provide guidance for the placement of the ultrasound probe 15 relative to the patient 22 through vibrational feedback (e.g., haptic feedback) provided to the operator through haptic feedback device 14. Such haptic feedback and guidance may appeal to an alternate sense perception than sight, which may provide more intuitive guidance to an operator instead of visual cues as the operator is already focused on viewing the ultrasound image, or voice guidance as the operator may be trying to communicate with the patient. For example, visual sense perception of the operator is already used to comprehend the live ultrasound image, to operate the workstation 16, to communicate with the patient 22, and in other events taking place near the ultrasound imaging system 10. Additionally, auditory sense perception is used for communicating with the patient 22. As tactile sense perception of the operator is already used for moving the ultrasound probe 15, such that the operator is not looking at the ultrasound probe 15, but rather is visually focused on the workstation 16 and the live ultrasound image, use of the vibration actuators 40 in the haptic feedback device 14 (e.g., worn or held by the hand of the operator) for probe positioning may take advantage of an underutilized sense perception. By using the actuator array 38 of the multiple vibration actuators 40, the position and orientation of the ultrasound probe 15 may be guided in all six degrees-of-freedom. As such, the haptic guidance system 12 may allow for more efficient and effective use of the ultrasound imaging system 10 by novice or less-skilled operators and thus, may increase operator independence of the ultrasound imaging system 10.

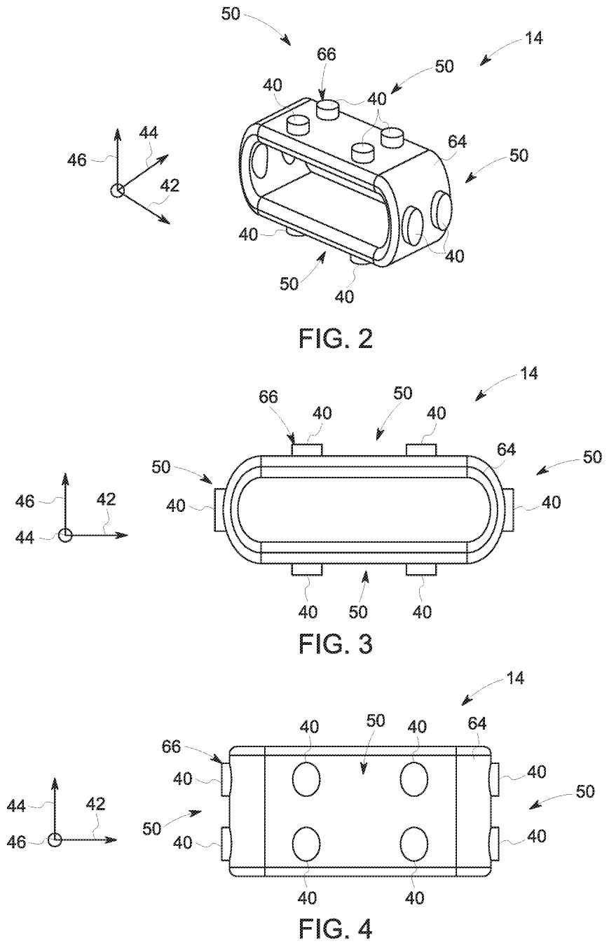

FIGS. 2-4 illustrate various views of the haptic feedback device 14 showing an embodiment of the placement of the vibration actuators 40 of the actuator array 38 of the haptic feedback device 14. For example, FIG. 2 illustrates a perspective view of the haptic feedback device 14. FIG. 3 illustrates a side view of the haptic feedback device 14 of FIG. 2. FIG. 4 illustrates an overhead view of the haptic feedback device 14 of FIGS. 2 and 3. To facilitate discussion, the haptic feedback device 14 and its components may be described with reference to a lateral axis or direction 42, a longitudinal axis or direction 44, and a vertical axis or direction 46.

As previously discussed, the vibration actuators 40 may be directed to vibrate in particular vibration patterns (e.g., actuation patterns or excitation patterns) representing particular movements of the ultrasound probe 15 relative to six degrees-of-freedom in which the ultrasound probe 15 can be moved. The vibration patterns of the vibration actuators 40 may guide the operator to position the ultrasound probe 15 in the appropriate position and/or orientation to sufficiently image the desired anatomy in the desired scan plane. The multiple vibration actuators 40 of the actuator array 38 may be distributed about the circumference of the haptic feedback device 14. That is, each side of the haptic feedback device 14 may include one or more vibration actuators 40 of the actuator array 38. Although the illustrated embodiment depicts the haptic feedback device 14 as having the vibration actuators 40 arranged in two rows about the circumference of the haptic feedback device 14, in other embodiments, the vibration actuators 40 may be arranged in one row, three rows, four rows, or any other suitable number of rows to convey guidance to the operator of the ultrasound probe 15. Additionally, although the illustrated embodiment depicts the haptic feedback device 14 as having twelve vibration actuators 40, in some embodiments, the haptic feedback device 14 may have two, three, four, five, six, seven, eight, nine, ten, sixteen, or any other suitable number of vibration actuators 40 for conveying guidance to the operator of the ultrasound probe 15.

In the illustrated embodiment, each side of the haptic feedback device 14 may form a band. For example, the operator of the ultrasound probe 14 may wear the band around a hand of the operator, an arm of the operator, one or more fingers of the operator, or any other suitable body part. As such, the haptic feedback device 14 may have any suitable shape and/or suitable size that corresponds to the body part of the operator that fits within the band. Although the illustrated embodiment depicts the haptic feedback device 14 as having an elliptical cross-section along the lateral axis 42 of the haptic feedback device 14, in other embodiments, the haptic feedback device 14 may have a circular, a triangular, a rectangular, a trapezoidal, a square, a diamond, a pentagonal, a hexagonal, a heptagonal, or any other suitable shaped cross-section along the lateral axis 42 of the haptic feedback device 14. In some embodiments, the haptic feedback device 14 may stretch around the body part of the operator that fits within the band. For example, the haptic feedback device 14 may include stretchable material such that the haptic feedback device 14 may fit around the finger of the operator or the hand of the operator. As will be appreciated, though the term “side” is used herein, this term should be understood broadly to encompass any facings or surfaces of the haptic feedback device 14. For example, in the context of a band having a circular cross-section, vibration actuators may be provided around the band such that different vibration actuators face different directions adjacent or opposite one another (i.e., face different radial directions with respect to the band), though no structurally distinct or discernible side may be geometrically identifiable.

The vibration actuators 40 may be integrated into the haptic feedback device 14. That is, the vibration actuators 40 may each be disposed above, below, or embedded within a housing 64 of the haptic feedback device 14. That is, although the vibration actuators 40 are illustrated as disposed outside of or on an outer surface of the housing 64 for visual clarity, the vibration actuators 40 may be disposed within the haptic feedback device 14 adjacent to the housing 64. In some embodiments, each vibration actuator 40 may be housed in a shell 66 such that each vibration actuator 40 is at least partially decoupled from the housing 64. For example, each vibration actuator 40 may be surrounded by a rubber ring, covering, or other suitable structure, such that the vibrations are spatially contained to the location of the individual vibration actuators 40 and minimal vibration is passed through the housing 64 and thus, providing location-specific vibrations. Location-specific vibration of each vibration actuator 40 may allow the operator to better sense the vibration patterns that may be used to guide the operator to position the ultrasound probe 15 and/or or haptic feedback device 14 into the appropriate position for capturing the desired scan plane of the desired anatomy (e.g., anatomy of interest).

In some embodiments, the guidance method may be varied, as may the actuation mechanisms. The embodiments described herein may facilitate positioning of the ultrasound probe 15 and/or haptic feedback device 14 based on haptic feedback in all six degrees-of-freedom (i.e., three denoting spatial position and three denoting orientation, such as angular orientation). In some embodiments, the concepts described herein with respect to the actuator array 38 of the vibration actuators 40 and various vibration patterns may be extended to other actuation mechanisms, such as small motion actuators oriented perpendicular to the haptic feedback device housing that protrude in and out at each actuation location, small motion actuators mounted in a shear orientation about the haptic feedback device housing (e.g., parallel with the housing) that drag across the hand of the operator, small electrical currents coupled into the hand through electrodes at each of these locations, small puffs of air at each of these locations, small protrusions in and out of a deformable membrane due to pressurization and depressurization of fluid filled chambers, or the like. As an example of the various possible guidance methods, a simpler haptic feedback approach may fuse visual feedback or voice commands with a single vibration that is provided as the ultrasound probe 15 and/or the haptic feedback device 14 is moved into the appropriate position. For example, a feedback pulse may become faster or slower as the ultrasound probe 15 and/or the haptic feedback device 14 is moved into the appropriate position. In some embodiments, the guidance method may include a fusion of visual feedback, auditory commands, and/or haptic feedback using the vibration actuators 40, where motion in some degrees-of-freedom may be indicated through haptic feedback and motion in other degrees-of-freedom may be indicated through visual and/or auditory mechanisms.

The controller 24 of the ultrasound imaging system 10 may cause vibration of the vibration actuators 40 integrated into the haptic feedback device 14 to guide the operator to move the ultrasound probe 15 into the appropriate position and/or orientation to image the desired anatomy in the desired scan plane. The particular vibration patterns (e.g., excitation patterns) may indicate to the operator how to move the ultrasound probe 15 to acquire an image of the desired anatomy in the desired scan plane. Particular vibration patterns may indicate to the operator to move the ultrasound probe 15 in a particular direction (e.g., left or right), to tilt or rock the ultrasound probe 15, to rotate the ultrasound probe 15, and/or to compress (e.g., apply pressure) or decompress (e.g., apply less pressure) the ultrasound probe 15 toward or away from the patient 22. In some embodiments, the operator may be guided to move the ultrasound probe 15 in more than one degree-of-freedom simultaneously by applying more complex vibration patterns.

For each vibration pattern discussed below, the vibrations of the vibration actuators 40 may continue until the operator has moved the ultrasound probe 15 and/or haptic feedback device 14 to the appropriate position. In some embodiments, to guide a speed of movement of the ultrasound probe 15 by the operator, a repetition rate of the vibration pattern may vary or modulate. For example, in some embodiments, the repetition rate may be faster when the ultrasound probe 15 is farther away from the appropriate position, and then may slow down as the ultrasound probe 15 is moved closer to the appropriate position to image the desired anatomy and/or the desired scan plane. In some embodiments, the repetition rate may be slower when the ultrasound probe 15 is farther away from the appropriate position, and then may increase in speed as the ultrasound probe 15 is moved closer to the appropriate position to image the desired anatomy and/or the desired scan plane. Additionally or alternatively, an intensity of the vibrations may vary. For example, in some embodiments, the vibrations may be stronger when the ultrasound probe 15 is farther away from the appropriate position, and then may decrease in intensity when the ultrasound probe 15 is moved closer to the appropriate position to image the desired anatomy and/or the desired scan plane. In some embodiments, the vibrations may be lower intensity when the ultrasound probe 15 is farther away from the appropriate position, and then may increase in intensity when the ultrasound probe 15 is moved closer to the appropriate position to image the desired anatomy and/or the desired scan plane. As such, the speed and/or the intensity of the vibrations of the vibration actuators 40 may operate as a closed feedback loop to more accurately and efficiently guide the operator to adjust the ultrasound probe 15 into the appropriate position.

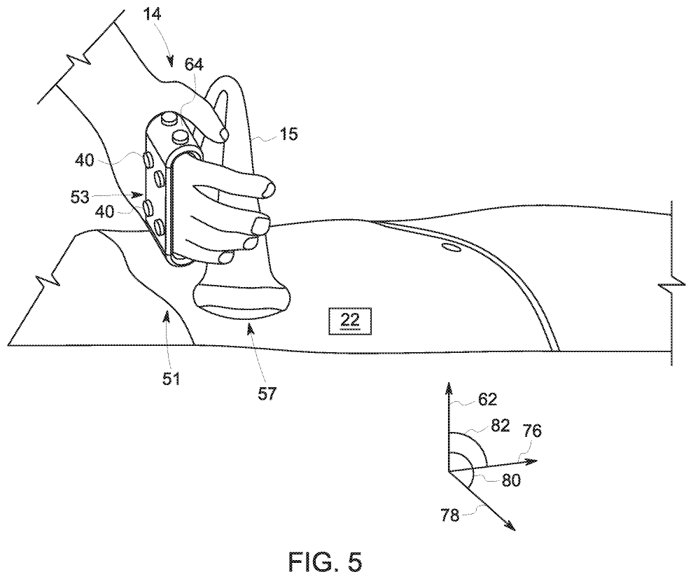

FIG. 5 illustrates a perspective view of the haptic feedback device 14 worn about a hand 51 of the operator 52 of an ultrasound probe 15. For example, the haptic feedback device 14 may include a band that may be worn about the palm and the back 53 of the hand 51 of the operator 52 being used to move or orient the ultrasound probe 15 and/or the patient-facing surface 57 of the ultrasound probe 15. As described above, the ultrasound imaging system 10 may determine the grip style of the operator's hand 51 on the ultrasound probe 15, and send a signal to the haptic feedback device 14 to provide one or more vibration patterns via the vibration actuators 40 to guide the operator in positioning and/or orienting the ultrasound probe 15 to acquire a desired scan plane of the desired anatomical feature of the patient 22. The particular vibration patterns may guide the operator to move the ultrasound probe 15 in six degrees-of-freedom, including right and left movement, forward and backward movement, compression and decompression (e.g., applying pressure or applying less pressure), tilt along an elevation direction, rock along an azimuth direction, and rotation. Additionally, movement of the ultrasound probe 15 in six degrees-of-freedom is discussed herein with reference to the axial axis or direction 62 (e.g., Z-axis), an X-axis 76, a Y-axis 78, an elevation angle 80, and an azimuth angle 82.

With the foregoing in mind, examples of the vibration patterns that may be used with the haptic feedback device 14 to guide particular movement of the ultrasound probe 15 are discussed below with reference to FIGS. 6-11. In the illustrated embodiment, the orientation of the haptic feedback device 14 is such that the haptic feedback device 14 fits around the hand of the operator while the operator is gripping, for example, the handle of the ultrasound probe 15 (e.g., FIG. 5). It should be noted that the vibration patterns may be used with other orientations of the haptic feedback device 14 and/or other grip styles utilized by the operator.

The particular vibration patterns may guide the operator to move the ultrasound probe 15 in six degrees-of-freedom, including right and left movement, forward and backward movement, compression and decompression (e.g., applying pressure or applying less pressure), tilt along an elevation direction, rock along an azimuth direction, and rotation. In FIGS. 6-11, the vibration patterns are shown as a series of vibration pulses, where open or non-filled vibration actuators 40 indicate still or currently non-vibrating actuators and solid or filled vibration actuators 40 indicate currently vibrating actuators. For reference, it should be noted that with regard to FIGS. 6-11, directional motion is discussed in terms of the operator being positioned within the page such that forward movement describes movement out of the page and backward movement describes movement into the page. For example, as illustrated in FIG. 5, the back 53 of the hand 51 of the operator 52 is facing to the left of the page along axis 76, and the palm of the operator is facing to the right of the page along axis 76. As described further herein, the vibration actuators 40 may be used to guide movement of the ultrasound probe 15 left or right along the X-axis 76 (e.g., toward a left side of the patient 22 or toward a right side of the patient 22), forward or backward along the Y-axis 78 (e.g., out of the page or into the page), or along the axial axis 62 (e.g., pressure toward the tissue of the patient 22 or less pressure away from the tissue of the patient 22). Additionally, the vibration actuators 40 may be used to guide a tilting movement of the ultrasound probe 15 forward or backward, thus increasing or decreasing the elevation angle 80 between the axial axis 62 and the Y-axis 78; guide rocking movement of the ultrasound probe 15 left or right along the azimuth direction to increase or decrease the azimuth angle 82 between the axial axis 62 and the X-axis 76; or guide a rotational motion of the ultrasound probe 15 in a circumferential direction 140 around the axial axis 62 (e.g., Z-axis) in a clockwise or counterclockwise direction. It should be noted that the vibration patterns and the corresponding directions and/or angles of movement discussed herein are based on the relative positions of the operator's hand 51 within the haptic feedback device 14 and the ultrasound probe 15 as illustrated in FIG. 5. Additionally, with regard to FIGS. 6-11, while the vibration patterns are described in terms of indicating movement toward the pulsing or longer vibrations or to follow the vibration pattern, in some embodiments, the vibration patterns may indicate movement away from the pulsing or longer vibration or to move opposite the vibration pattern based on operator preferences

FIG. 6 illustrates an embodiment of a simple vibration pattern (denoted by iteration between the left-hand and right-hand depictions, as denoted by the two-way arrow) of the vibration actuators 40 that may be used to guide movement of the ultrasound probe 15 along the X-axis 76. For example, the illustrated vibration pattern may be used to guide movement of the ultrasound probe 15 in a linear direction e.g., toward a first side 58 (e.g., right side). That is, the haptic guidance system 12 may cause the vibration actuators 40 on a first half 84 (e.g., a first half about the axial axis 62 extending from the patient-facing surface 54 to an opposite non-patient-facing surface 55) of the haptic feedback device 14 to pulse, as shown between the left-hand and right-hand depictions of FIG. 6. Such on-and-off pulsing of the vibration actuators 40 may indicate to the operator of the ultrasound probe 15 to move the ultrasound probe 15 in the direction of the pulsing. In some embodiments, the pulses of the vibration actuators 40 on the first half 84 of the haptic feedback device 14 may be short pulses, long pulses, or a combination thereof

In some embodiments, the pulse length, pulse repetition rate, and/or pulse intensity may vary to indicate a distance from and/or to guide a speed of the movement of the ultrasound probe 15 to the appropriate position and/or orientation to sufficiently image the desired anatomy in the desired scan plane. For example, the pulse repetition rate of the vibration actuators 40 on the first half 84 of the haptic feedback device 14 may be faster when the ultrasound probe 15 is farther away from the appropriate position, and then may slow down as the ultrasound probe 15 is moved closer to the appropriate position to image the desired anatomy and/or the desired scan plane, or vice versa. Additionally or alternatively, an intensity of the vibrations may be stronger when the ultrasound probe 15 is farther away from the appropriate position, and then may decrease in intensity when the ultrasound probe 15 is moved closer to the appropriate position to image the target anatomy and/or the target scan plane, or vice versa. Further, additionally or alternatively, the pulse length of the vibrations of the vibration actuators 40 of the first half 84 of the haptic feedback device 14 may be longer when the ultrasound probe 15 is farther away from the appropriate position, and then may decrease in length when the ultrasound probe 15 is moved closer to the appropriate position to image the desired anatomy and/or the desired scan plane, or vice versa. Thus, the simple pulsing of the vibration actuators 40 of the first half 84 of the haptic feedback device 14 caused by the controller 24 of the haptic guidance system 12 may guide the operator of the ultrasound probe 15 to move the ultrasound probe 15 in the direction of the pulsing along the X-axis 76.

The opposite vibration pattern may be used to guide movement of the ultrasound probe 15 in the opposite direction along the X-axis 76. That is, the haptic guidance system 12 may cause the vibration actuators 40 on a second half 86 (e.g., a half opposite the first half about the axial axis 62 extending from the patient-facing surface 54 to an opposite non-patient-facing surface) of the haptic feedback device 14 to pulse, indicating to the operator of the ultrasound probe 15 to move the ultrasound probe 15 toward a second side 86 (e.g., left side) of the ultrasound probe 15 in the direction of the pulses. In some embodiments, the pulse length, pulse repetition rate, and/or pulse intensity may vary as discussed above to indicate a distance from and/or to guide a speed of the movement of the ultrasound probe 15 to the appropriate position to sufficiently image the desired anatomy in the desired scan plane.

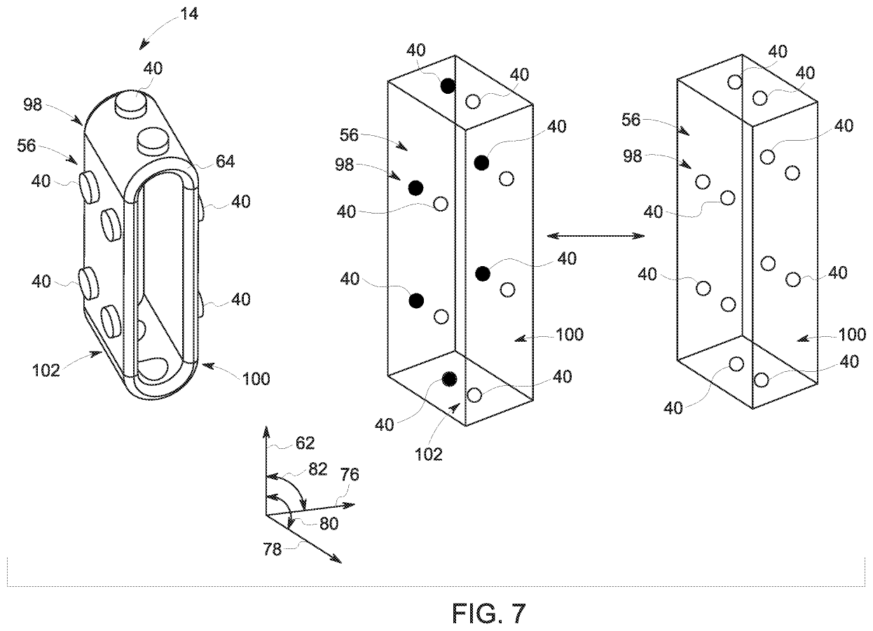

Similar vibration patterns may be used with the haptic feedback device 14 to guide movement of the ultrasound probe 15 forward (e.g., away from the operator) and backward (e.g., toward the operator) along the Y-axis 78, as illustrated in FIG. 7. For example, the illustrated vibration pattern may be used to guide movement of the ultrasound probe 15 backward toward the operator and toward the back side 56 of the haptic feedback device 14. The haptic guidance system 12, in some embodiments, may cause the vibration actuators 40 on a back half 98 of the haptic feedback device 14 to pulse, indicating to the operator of the ultrasound probe 14 to move the ultrasound probe 15 toward the back side 56 of the haptic feedback device 14 in the direction of the pulses. In some embodiments, the pulse length, pulse repetition rate, and/or pulse intensity may vary, as discussed above with reference to FIG. 6, to indicate a distance from and/or to guide a speed of the movement of the ultrasound probe 15 to the appropriate position to sufficiently image the target anatomy in the target scan plane.

The opposite vibration pattern may be used to guide movement of the ultrasound probe 14 in the opposite direction (e.g., forward) away from the operator and toward a front side 100 (e.g., side opposite the back side) of the haptic feedback device 14 along the Y-axis 78. That is, the haptic guidance system 12 may cause the vibration actuators 40 on a front half 102 of the haptic feedback device 14 to pulse, indicating to the operator of the ultrasound probe 15 to move the ultrasound probe 15 forward toward the front side 100 of the haptic feedback device 14 in the direction of the pulses. In some embodiments, the pulse length, pulse repetition rate, and/or pulse intensity may vary as discussed above to indicate a distance from and/or to guide a speed of the movement of the ultrasound probe 15 to the appropriate position to sufficiently image the target anatomy in the target scan plane.

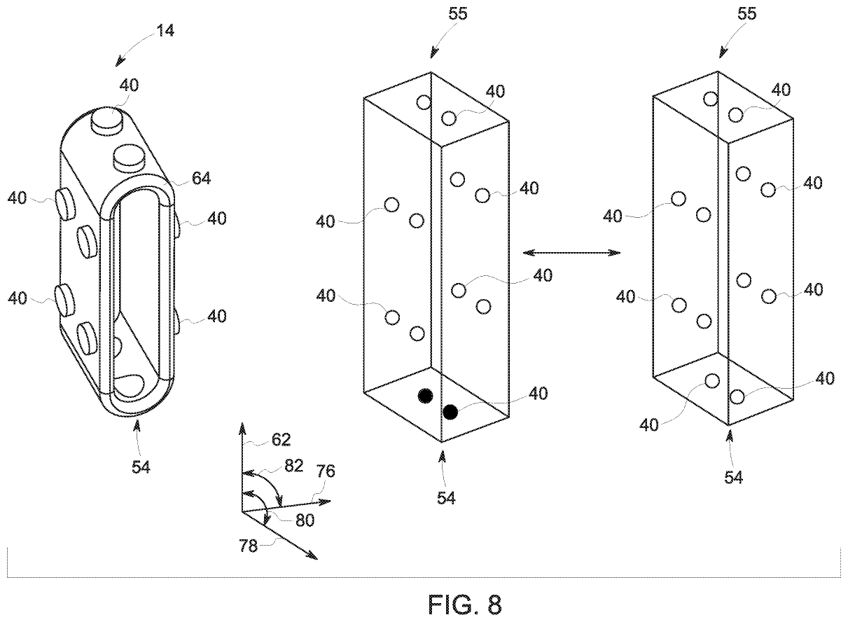

Similar vibration patterns may be used to guide compression and decompression of the ultrasound probe 15 along the axial axis 62 (e.g., Z-axis), as illustrated in FIG. 8. As used herein, the term “compression” may refer to applying pressure to the ultrasound probe 15 such that the ultrasound probe 15 moves toward the tissue of the patient 22, and the term “decompression” may refer to applying less pressure to the ultrasound probe 15 such that the ultrasound probe 15 is moved in a direction away from the tissue of the patient 22. For example, the illustrated vibration pattern may be used with the haptic feedback device 14 to guide compression movement of the ultrasound probe 15 toward the tissue of the patient 22 and toward the patient-facing surface 54 of the haptic feedback device 14. The haptic guidance system 12, in some embodiments, may cause the vibration actuators 40 on the patient-facing surface 54 of the haptic feedback device 14 to pulse, indicating to the operator of the ultrasound probe 15 to move the ultrasound probe 15 toward the tissue of the patient 22 in the direction of the pulses. In some embodiments, the pulse length, pulse repetition rate, and/or pulse intensity may vary, as discussed above with reference to FIG. 6, to indicate a distance from and/or to guide a speed of the movement of the ultrasound probe 15 to the appropriate position to sufficiently image the desired anatomy in the desired scan plane.

The opposite vibration pattern may be used with the haptic feedback device 14 to guide decompression movement of the ultrasound probe 15 in the opposite direction away from the tissue of the patient 22 and toward a non-patient-facing surface 55 (e.g., surface opposite the patient-facing surface). That is, the haptic guidance system 12 may cause the vibration actuators 40 on the non-patient-facing surface 55 of the haptic feedback device 14 to pulse, indicating to the operator of the ultrasound probe 15 to move the ultrasound probe 15 away from the tissue of the patient 22 in the direction of the pulses. In some embodiments, the pulse length, pulse repetition rate, and/or pulse intensity may vary, as discussed above, to indicate a distance from and/or to guide a speed of the movement of the ultrasound probe 15 to the appropriate position to sufficiently image the target anatomy in the target scan plane.

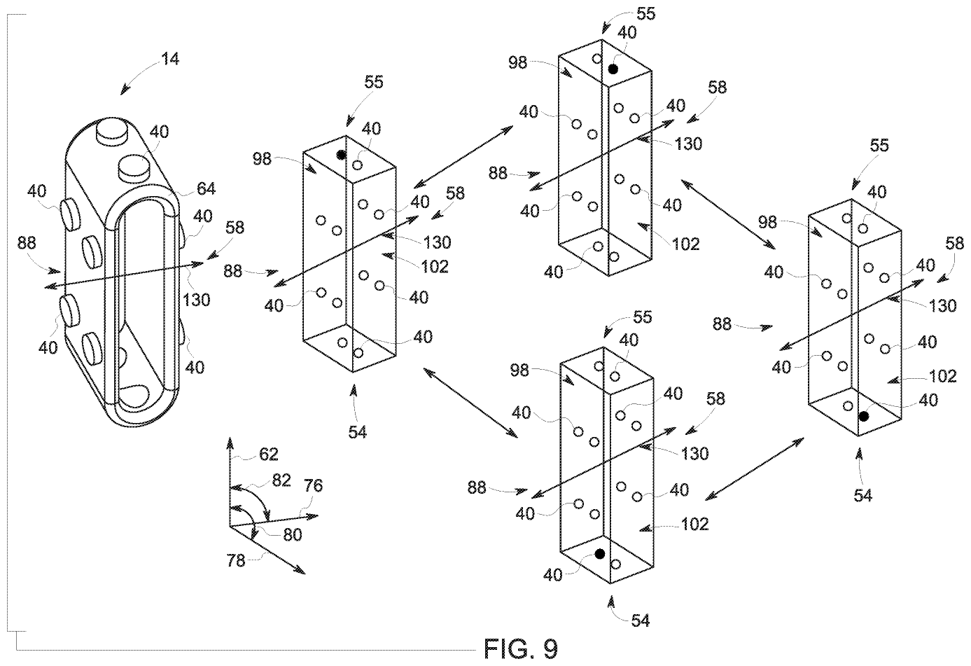

To guide orientation of the patient-facing surface of the ultrasound probe 15, and thus the transducer array 18, within the three other degrees-of-freedom via the haptic feedback device 14, circular vibration patterns may be used, which the operator may follow to adjust the orientation of the ultrasound probe 15 into a desired orientation. For example, FIG. 9 illustrates an embodiment of a vibration pattern that may be used with the haptic feedback device 14 to guide a tilting movement of the ultrasound probe 15 forward or backward along the elevation direction to increase or decrease the elevation angle 80. As such, the illustrated vibration pattern may be used with the haptic feedback device 14 to guide a tilting movement of the ultrasound probe 15 such that the front side of the ultrasound probe 15 is moved closer to or farther from the patient 22, thus increasing or decreasing the elevation angle 80 between the axial axis 62 and the Y-axis 78 (e.g., between the front side of the ultrasound probe 15 and the patient 22).

In the illustrated embodiment, the vibration pattern may be used with the haptic feedback device 14 to guide a tilting movement of the ultrasound probe 15 forward, thus decreasing the elevation angle 80 between the patient 22 and the front side of the ultrasound probe 15. In some embodiments, the haptic guidance system 12 may send a signal to the haptic feedback device 14 to cause a vibration actuator 40 of the back half 98 of the non-patient-facing surface 55 to pulse, then cause a vibration actuator of the front half 102 of the non-patient facing surface 55 to pulse, then cause a vibration actuator 40 of the front half 102 of the patient-facing surface 54 to pulse, and then cause a vibration actuator 40 of the back half 98 of the patient-facing surface 54 to pulse, thus creating a circular vibration pattern.

In this manner, the circular vibration pattern may rotate around an axis 130 of the haptic feedback device 14 extending from the second side 88 to the first side 58 of the haptic feedback device 14. This circular pattern may indicate to the operator to follow the circular pattern to tilt the handle of the ultrasound probe 15 to decrease the elevation angle 80 between the front side of the ultrasound probe 15 and the patient 22, and thus cause the patient-facing surface 57 of the ultrasound probe 15 to tilt forward. In some embodiments, the pulse length, pulse repetition rate, and/or pulse intensity may vary, as discussed above with reference to FIG. 6, to indicate a distance from and/or to guide a speed of the movement of the ultrasound probe 15 to the appropriate orientation to sufficiently image the desired anatomy in the desired scan plane.

The opposite vibration pattern may be used to guide a tilting movement of the handle of the ultrasound probe 15 backward, thus increasing the elevation angle 80 between the patient 22 and the front side of the ultrasound probe 15. In some embodiments, the haptic guidance system 12 may send a signal to the haptic feedback device 14 to cause a vibration actuator 40 of the front half 102 of the non-patient facing surface 55 to pulse, then cause a vibration actuator 40 of the back half 98 of the non-patient facing surface 55 to pulse, then cause a vibration actuator 40 of the back half 98 of the patient-facing surface 54 to pulse, then cause a vibration actuator 40 of the front half 102 of the patient-facing surface 54 to pulse, thus creating a circular vibration pattern.

In this manner, the circular vibration pattern may rotate in the opposite direction around the axis 130 of the haptic feedback device 14 extending from the second side 88 to the first side 58 of the haptic feedback device 14. This circular pattern may indicate to the operator to follow the circular pattern to tilt the handle of the ultrasound probe 15 to increase the elevation angle 80 between the front side of the ultrasound probe 15 and the patient 22, and thus cause the patient-facing surface 57 of the ultrasound probe 15 to tilt backward. In some embodiments, the pulse length, pulse repetition rate, and/or pulse intensity may vary, as discussed above, to indicate a distance from and/or to guide a speed of the movement of the ultrasound probe 15 to the appropriate orientation to sufficiently image the target anatomy in the target scan plane.

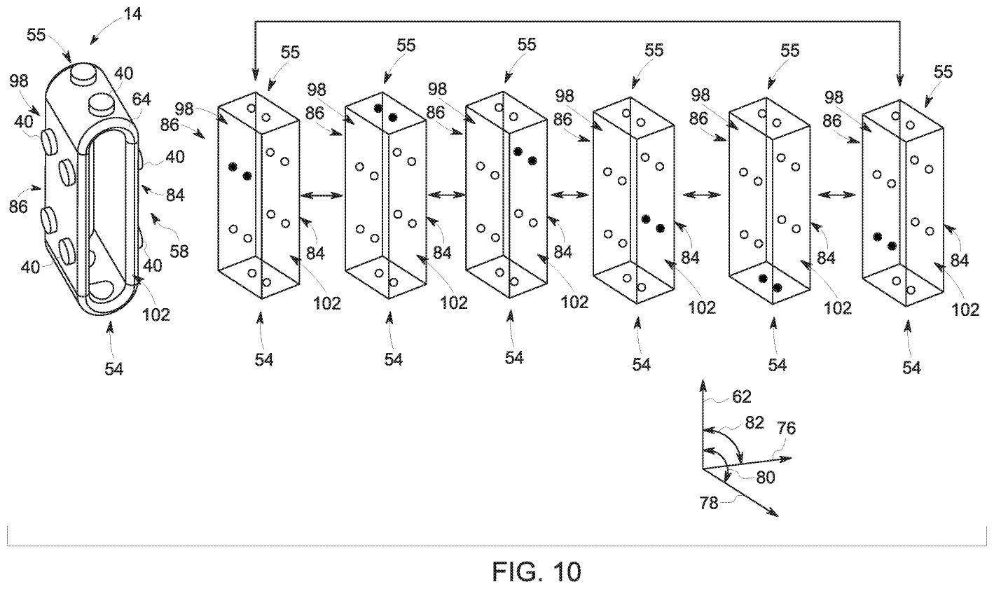

Similarly, FIG. 10 illustrates an embodiment of a vibration pattern that may be used to guide rocking movement of the ultrasound probe 15 left or right along the azimuth direction to increase or decrease the azimuth angle 82 (e.g., horizontal angle). For example, the illustrated vibration pattern may be used to guide rocking movement of the handle of the ultrasound probe 15 toward the first direction (e.g., right) such that that first side 58 is moved closer to the tissue of the patient 22, thus decreasing the azimuth angle 82 between the axial axis 62 and the X-axis 76 (e.g., between the first side of the ultrasound probe 15 and the patient 22). In some embodiments, the haptic guidance system 12 may cause the vibration actuators 40 of the upper row of the second half 86 of the haptic feedback device 14 to pulse, then cause the vibration actuators 40 of the non-patient-facing surface 55 of the haptic feedback device 14 to pulse, then the vibration actuators 40 of the upper row of the first half 86 of the haptic feedback device 14 to pulse, then cause the vibration actuators 40 of the lower row of the first half 86 of the haptic feedback device 14 to pulse, and then cause the vibration actuators 40 of the patient-facing surface 54 to pulse, and then cause the vibration actuators 40 of the lower row of the second half 86 of the haptic feedback device 14 to pulse, thus creating a circular vibration pattern.

In this manner, the circular vibration pattern may rotate around the Y-axis 78 of the haptic feedback device 14 extending from the front half 102 to the back half 98 of the haptic feedback device 14. This circular pattern may indicate to the operator to rock the handle of the ultrasound probe 15 toward the first direction to decrease the azimuth angle 82 between the first side of the ultrasound probe 15 and the patient 22, and thus cause the patient-facing surface 57 of the ultrasound probe 15 to face more toward the second direction. In some embodiments, the pulse length, pulse repetition rate, and/or pulse intensity may vary, as discussed above with reference to FIG. 6, to indicate a distance from and/or to guide a speed of the movement of the ultrasound probe 15 to the appropriate orientation to sufficiently image the desired anatomy in the desired scan plane.

The opposite vibration pattern may be used to guide rocking movement of the handle the ultrasound probe 15 in the opposite direction toward the second direction (e.g. left), thus increasing the azimuth angle 82 between the first side of the ultrasound probe 15 and the patient 22 and decreasing the azimuth angle 82 between the second side of the ultrasound probe 15 and the patient 22. That is, the haptic guidance system 12 may cause the vibration actuators 40 of the non-patient-facing surface 55 of the haptic feedback device 14 to pulse, the vibration actuators 40 of the upper row of the second half 86 of the haptic feedback device 14 to pulse, then cause the vibration actuators 40 of the lower row of the second half 86 of the haptic feedback device 14 to pulse, then cause the vibration actuators 40 of the patient-facing surface 54 to pulse, then cause the vibration actuators 40 of the lower row of the first half 86 of the haptic feedback device 14 to pulse, then cause the vibration actuators 40 of the upper row of the first half 86 of the haptic feedback device 14 to pulse, thus creating a circular vibration pattern.

In this manner, the circular vibration pattern may rotate around the Y-axis 78 of the haptic feedback device 14 extending from the front half 102 to the back half 98 of the haptic feedback device 14. As discussed above, the pulse speed and/or intensity may be varied as the ultrasound probe 15 is moved closer to the desired position to indicate proximity and/or speed of movement to the operator. In some embodiments, the pulse length, pulse repetition rate, and/or pulse intensity may vary, as discussed above, to indicate a distance from and/or to guide a speed of the movement of the ultrasound probe 15 to the appropriate orientation to sufficiently image the desired anatomy in the desired scan plane.

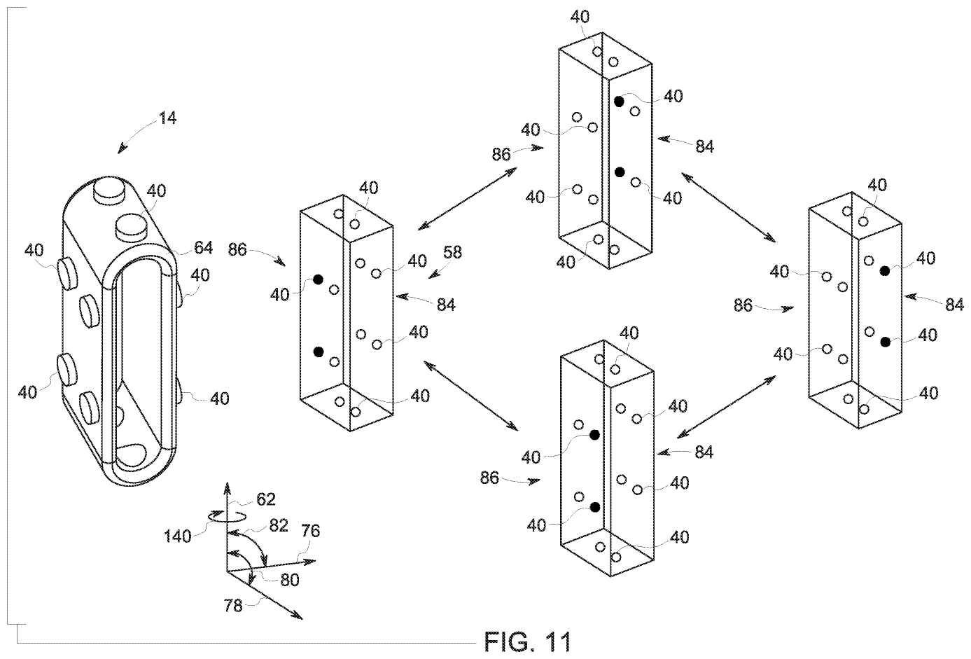

To cause a rotational motion of the ultrasound probe 15 in a circumferential direction 140 around the axial axis 62 (e.g., Z-axis) in a clockwise or counterclockwise direction, the haptic guidance system 12 may cause the vibration actuators 40 to vibrate in a circular pattern, as illustrated in FIG. 11. In some embodiments, the haptic guidance system 12 may send a signal to the haptic feedback device 14 to cause a back set of axially aligned vibration actuators 40 (e.g., a vibration actuator 40 in the lower row aligned with a vibration actuator in the upper row) of the second half 86 of the haptic feedback device 14 to pulse, then cause a back set of axially aligned vibration actuators 40 of the first half 84 of the haptic feedback device 14 to pulse, then cause a front set of axially aligned vibration actuators 40 of the first half 84 of the haptic feedback device 14 to pulse, then cause a front set of axially aligned vibration actuators 40 of the second half 86 of the haptic feedback device 14 to pulse, thus creating a circular vibration pattern in the clockwise direction. The operator may follow the pattern of the vibration to rotate the ultrasound probe 15, and thus the patient-facing surface 57 of the ultrasound probe 15, clockwise about the axial axis 62. In this manner, the circular vibration pattern may rotate around the axial axis 62 (e.g., Z-axis) of the ultrasound probe 15.

The opposite vibration pattern, may be used to guide rotational movement of the ultrasound probe 15 counterclockwise about the axial axis 62. For example, the haptic guidance system may send a signal to the haptic feedback device 14 to cause a back set of axially aligned actuators 40 of the second half 86 of the haptic feedback device 14 to pulse, then cause a front set of axially aligned actuators of the second half 86 of the haptic feedback device 14 to pulse, then cause a front set of axially aligned actuators 40 of the first half of the haptic feedback device 14 to pulse, then cause a back set of axially aligned actuators 40 of the first half of the haptic feedback device 14 to pulse, thus creating a circular vibrational pattern in the counter-clockwise direction. The operator may follow the pattern of the vibration to rotate the ultrasound probe 15, and thus the patient-facing surface 57 of the ultrasound probe 15, counter-clockwise about the axial axis 62. In some embodiments, the pulse length, pulse repetition rate, and/or pulse intensity may vary, as discussed above with reference to FIG. 6, to indicate a distance from and/or to guide a speed of the movement of the ultrasound probe 15 to the appropriate orientation to sufficiently image the target anatomy in the target scan plane.

The examples discussed above are vibration patterns that may be used with the haptic feedback device 14 to guide the operator to position and/or orient the ultrasound probe 15 to capture the desired scan plane of the desired anatomy. The examples of the vibration patterns discussed above are not intended to be limiting, but only as examples of possible vibration, or excitation, patterns. Other vibration patterns may encode or indicate suggested motion, and may be used to guide movement of the ultrasound probe 15. For example, the example vibration patterns discussed above may be implemented spatially on the haptic feedback device 14, such that the vibration patterns approximately align with the natural spatial locations (e.g., left, right, up, down, forward, backward, rotate). However, in some embodiments, motion direction information may be indicated directly in the vibration patterns with less emphasis on the spatial orientation of the vibration actuators 40. That is, in some embodiments, all of the vibration actuators 40 of the haptic feedback device 14 or a portion of the vibration actuators 40 may vibrate in a particular pattern to indicate movement in a particular direction or degree-of-freedom. For example, in one embodiment, all of the vibration actuators 40 of the haptic feedback device 14 may vibrate in a short-short-long-rest pattern and then repeat to indicate suggested compression movement toward the patient 22. Additionally, all of the vibration actuators 40 of the haptic feedback device 14 may vibrate in a long-short-short-rest pattern and then repeat to indicate suggested decompression movement away from the patient 22. Such vibration patterns do not rely on the spatial orientation of the vibration actuators 40 and variations of such vibration patterns maybe extrapolated to indicate motion in all six degrees-of-freedom. Additionally or alternatively, vibration patterns that do not rely on the spatial orientation of the vibration actuators 40 may be used in combination with the vibration patterns described in FIGS. 6-11 to convey suggested movement of the ultrasound probe 15 to guide the operator to move the probe to the appropriate position and orientation to image the desired anatomy in the desired scan plane, as different operators may be more receptive to different vibration pattern types.

Further, as described above, the ultrasound imaging system 10 may determine the grip style of the operator's hand 51 on the ultrasound probe 15. For example, the ultrasound imaging system 10 may determine that the operator is holding the ultrasound probe 15 in the hand 51 of the operator with a pen-like grip, a hammer grip, or the like. The ultrasound imaging system 10 may then send a signal to the haptic guidance system 12 indicative of the grip style determination such that the haptic guidance system 12 may provide corresponding vibrational feedback to the operator based on the determined grip style. In some embodiments, vibrational feedback provided to the operator via the haptic feedback device 14 is different based the grip style used by the operator on the ultrasound probe 15. For example, the haptic guidance system 12 may adjust the particular vibrational actuators 40 that pulse based on the grip style used by the operator such that the operator may discern a command to move and/or orient the ultrasound probe 15 regardless of which grip style used.

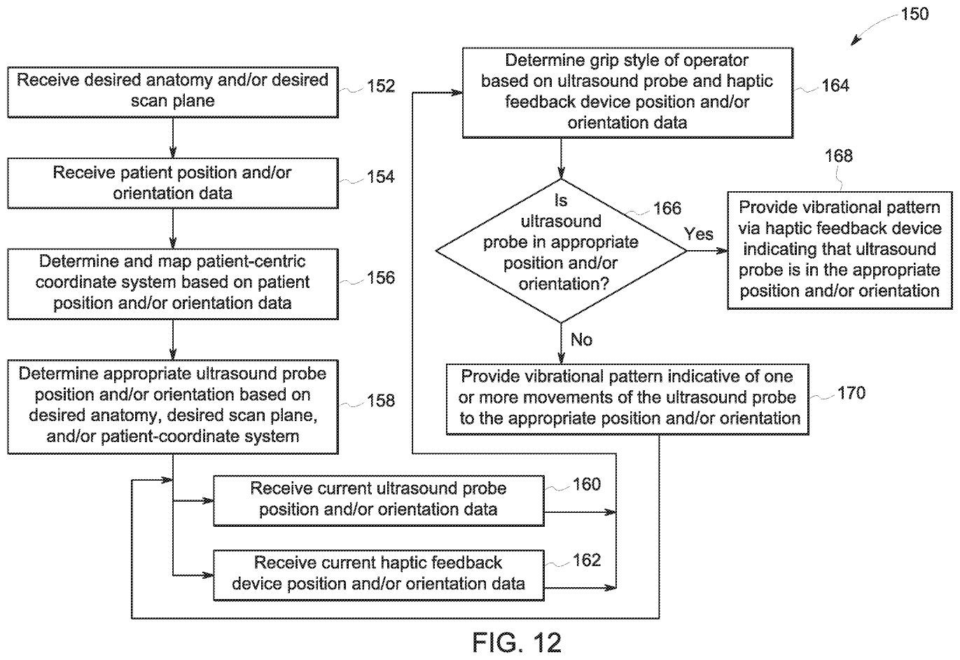

With the foregoing in mind, FIG. 12 illustrates a flow chart of a method 150 for guiding movement of the ultrasound probe 15 via the haptic feedback device 14 to position the ultrasound probe 15 in the appropriate position and/or orientation to sufficiently image the desired anatomy in the desired scan plane. Although the following description of the method 150 is described in a particular order, it should be noted that the method 150 is not limited to the depicted order, and instead, the method 150 may be performed in any suitable order. Moreover, although the method 150 is described as being performed by the controller 24 of the haptic guidance system 12, it should be noted that it may be performed by any suitable computing device.

At step 152, the controller 24 of the haptic guidance system 12 may receive the desired anatomy and/or the desired scan plane to be imaged. For example, the desired anatomy in the desired scan plane may be a user input via the haptic guidance system 12. At step 154, the controller 24 may receive position and/or orientation data of the patient 22 via the patient position sensor 33. That is, the controller 24 may receive one or more signals from the patient position sensor 33 indicative of the position of the patient 22 about the imaging space 35. Additionally or alternatively, the position of the patient 22 may be determined based on live ultrasound image data acquired via the ultrasound probe 15. At step 156, the controller 24 may determine and map a patient-centric coordinate system based at least in part on the received patient position. For example, the patient-centric coordinate system may include an anatomical atlas of the patient 22 that may be mapped to anatomy of the patient 22. In one embodiment, the patient-centric coordinate system for the patient 22 may be saved in the memory 32. In another embodiment, this saved coordinate system may be updated based on the current position of the patient 22. At step 158, the controller 24 may determine the appropriate position and/or orientation of the ultrasound probe 15 based at least in part on the desired anatomy, the desired scan plane, the patient-centric coordinate system, and/or live ultrasound image data of the patient 22. In some embodiments, the desired anatomy and/or the desired scan plane in a set of live ultrasound image data may be identified, and an appropriate ultrasound probe position may derive a desired ultrasound probe position and/or orientation to acquire the desired anatomy and/or the desired scan plane in subsequently acquired ultrasound image data.

At step 160, the controller 24 may receive current position data and/or orientation data associated with the ultrasound probe 15 from the probe position sensor 36. In some embodiments, the controller 24 may receive current position data and/or orientation data associated with the ultrasound probe 15, live ultrasound image data acquired via the ultrasound probe 15, or both. As described herein, the controller 24 may use the live ultrasound image data to determine whether the ultrasound probe 15 is in the appropriate position and/or orientation to acquire imaging data of the desired anatomy in the desired scan plane of the patient 22.

At step 162, the controller 24 may also receive current position data and/or orientation data associated with the haptic feedback device 14 from the haptic feedback device position sensor 37. For example, the controller 24 may receive position data and/or orientation data from one or more inertial measurement units, one or more gyroscopes, one or more magnetometers, or the like, associated with the haptic feedback device 14.

After receiving current position data and/or orientation data of the ultrasound probe 15, the haptic feedback device 14, or both, at step 164, the controller 24 may determine a grip style used by the operator to maneuver or hold the ultrasound probe 15 with the hand of the operator. As described herein, for example, the position and/or orientation data of the ultrasound probe 15 may include a plurality of probe reference points (e.g., x-coordinate, y-coordinate, z-coordinate) relative to the imaging space 35, and the position and/or orientation data of the haptic feedback device 14 may include a plurality haptic feedback device reference points (e.g., x-coordinate, y-coordinate, z-coordinate) relative to the imaging space 35. The controller 24 may compare the relative positions of the probe reference points and the haptic feedback device reference points in the imaging space 35 to lookup tables stored in the memory 32 to determine the grip style used by the operator on the ultrasound probe 15.

After determining the grip style used by the operator with the ultrasound probe 15, the controller 24 may determine whether the position and/or orientation of the ultrasound probe 15 corresponds to the appropriate position and/or orientation to image the desired anatomy in the desired scan plane at step 166. In some embodiments, the controller may determine whether the position and/or orientation of the ultrasound probe 15 correspond to the appropriate position and/or orientation based at least in part on the patient-centric coordinate system, the position data and/or the orientation data of the ultrasound probe 15, live ultrasound imaging data, the position data and/or the orientation data of the haptic feedback device 14, or a combination thereof

For example, the controller 24 may use the position data and/or orientation data of the ultrasound probe 15, the position data and/or orientation data of the haptic feedback device 14, and/or live ultrasound image data captured by the ultrasound probe 15 as feedback to determine whether the ultrasound probe 15 is in the appropriate position and/or orientation to acquire ultrasound imaging data of the desired anatomy in the desired scan plane. If the controller 24 determines that the ultrasound probe 15 is in the appropriate position and/or orientation to sufficiently image the desired anatomy in the desired scan plane, the method 150 may continue to step 168 in which the controller 24 may send a signal to the vibration actuators 40 of the haptic feedback device 14 to pulse at a particular pulse pattern indicating that the ultrasound probe 15 is in the appropriate position and/or orientation to the operator. In some embodiments, the controller 24 may cause display of a notification, such as a visual or audible notification in addition to providing vibrational feedback to the operator. Additionally or alternatively, the controller 24 may cause automatic storage of the live ultrasound image data in the memory 32 of the ultrasound imaging system 10 after the controller 24 has determined that the ultrasound probe 15 is in the appropriate position and/or orientation.

Referring back to step 166, if the controller 24 determines that the ultrasound probe 15 is not in the appropriate position and/or orientation to sufficiently image the desired anatomy in the desired scan plane, the controller 24 may send a signal to the vibration actuators 40 of the haptic feedback device 14 to pulse at one or more vibration patterns indicative of one or more movements of the ultrasound probe 15 to position and/or orient the ultrasound probe 15 in the appropriate position at step 170. For example, the controller 24 may send a signal indicative of one or more vibration patterns as described herein with regard to FIGS. 6-11. The controller 24 may determine which vibration patterns to provide to the operator via the haptic feedback device 14 based at least in part on the patient-centric coordinate system, the position and/or orientation of the ultrasound probe 15, live ultrasound image data, the position and/or orientation of the haptic feedback device 14, the grip style used by the operator, or a combination thereof.

The vibration pattern provided to the operator via the haptic feedback device 14 may indicate to the operator of the ultrasound probe 15 to move the ultrasound in a particular direction or manner to achieve the appropriate position and/or orientation to sufficiently image the desired anatomy in the desired scan plane. The controller 24 may then continue to repeat steps 160, 162, 164, 166, and 170 until the controller 24 determines that the ultrasound probe 15 is in the appropriate position and/or orientation to sufficiently image the desired anatomy in the desired scan plane. After determining that the ultrasound probe 15 is in the appropriate position and/or orientation to sufficiently image the desired anatomy in the desired scan plane, the method 150 may proceed to step 168, as described herein.

The haptic guidance system 12 disclosed provides positioning guidance of the ultrasound probe 15 by utilizing an alternate sense perception of the operator (e.g., tactile sense perception). Such haptic feedback may provide more intuitive guidance to the operator instead of visual cues, or voice guidance, as the operator is already focused on viewing the ultrasound machine and communicating with the patient 22. Additionally, an array of vibration actuators 40 associated with a haptic feedback device 14 may facilitate operator guidance of the ultrasound probe 15 in all six degrees-of-freedom and may accommodate multiple grip styles. In some embodiments, the haptic feedback device 14 may be worn by the operator such that the operator may discern vibration feedback from the haptic feedback device 14 in response to the current position and/or orientation of the ultrasound probe in relation to the desired anatomy and/or the desired scan plane to be imaged. Further, in the big picture, an operator-independent ultrasound imaging system, such as the system described above including the haptic guidance system, may allow novice users to successfully image the desired anatomy in the desired scan plane.

While only certain features of the present disclosure have been illustrated and described herein, many modifications and changes will occur to those skilled in the art. It is, therefore, to be understood that the appended embodiments are intended to cover all such modifications and changes as fall within the scope of the disclosure.

1. An ultrasound imaging system, comprising:
an ultrasound probe;
a monitor comprising a memory and a processor, wherein the processor is communicatively coupled to the ultrasound probe and a haptic feedback device comprising a plurality of haptic actuators, and the processor is configured to:
determine a current position of the ultrasound probe relative to a region of interest of a patient, wherein the region of interest comprises a desired anatomy, a desired scan plane, or both, to be imaged via the ultrasound probe;
determine whether the current position of the ultrasound probe corresponds to a desired position of the ultrasound probe relative to the region of interest, wherein the desired position of the ultrasound probe facilitates an acquisition of an ultrasound image of the region of interest; and
in response to determining that the current position of the ultrasound probe does not correspond to the desired position of the ultrasound probe, transmitting a control signal to the plurality of haptic actuators, wherein the control signal causes the plurality of haptic actuators to activate in an actuation pattern indicative of a suggested movement of the ultrasound probe to position the ultrasound probe in the desired position.

2. The ultrasound imaging system of claim 1, wherein the haptic feedback device is configured to be worn an operator of the ultrasound probe about a body part of the operator.

3. The ultrasound imaging system of claim 1, wherein the processor is configured to determine a current orientation of the ultrasound probe and a current orientation of the haptic feedback device, and determine a grip style used by an operator of the ultrasound probe based on the current orientation of the ultrasound probe and the current orientation of the haptic feedback device.

4. The ultrasound imaging system of claim 3, wherein the processor is configured to determine the actuation pattern based on the grip style used by the operator of the ultrasound probe, and wherein transmitting the control signal to the plurality of haptic actuators is based on the determined actuation pattern.

5. The ultrasound imaging system of claim 1, wherein the haptic actuators comprise vibration motors, motion actuators, electrodes, or deformable fluid-filled chambers.

6. The ultrasound imaging system of claim 1, wherein the actuation pattern is indicative of one or more suggested movements in six degrees-of-freedom.

7. The ultrasound imaging system of claim 6, wherein the one or more suggested movements comprise a directional movement, a rotational movement, a tilting movement, a compression, a decompression, or a combination thereof.

8. The ultrasound imaging system of claim 1, wherein the processor is configured to:
determine an additional current position of the ultrasound probe relative to the region of interest after the ultrasound probe has been moved from the current position;
determine whether the additional current position of the ultrasound probe corresponds to the desired position of the ultrasound probe; and
transmit an additional control signal to the plurality of haptic actuators in response to determining that the additional current position of the ultrasound probe corresponds to the desired position of the ultrasound probe, and wherein the control signal causes the plurality of vibrational actuators to actuate in an additional actuation pattern indicative of the determination that the additional current position corresponds to the desired position.

9. A wearable device for providing ultrasound guidance feedback, comprising:
a housing comprising a plurality of haptic actuators; and
an opening extending along an axis through the housing, wherein the opening is configured to fit about a body part of an operator of an ultrasound probe, and wherein the haptic actuators are configured to pulse in an actuation pattern indicative of one or more suggested movements of the ultrasound probe to position the ultrasound probe in a desired position to acquire imaging data of a region of interest of a patient.

10. The wearable device of claim 9, wherein the body part of the operator is a hand of the operator, and the wearable device is a glove or a band configured to fit about the hand of the operator.

11. The wearable device of claim 9, wherein the haptic feedback device comprises one or more accelerometers, one or more gyroscopes, one or more magnetometers, one or more inertial measurement units, or a combination thereof.

12. The wearable device of claim 9, wherein the plurality of haptic actuators are disposed above the surface of the housing, below the surface of the housing, or embedded within the housing.

13. The wearable device of claim 9, wherein the plurality of haptic actuators comprise a plurality of axially aligned rows of haptic actuators that are spatially disposed about the circumference of the housing.

14. The wearable device of claim 9, wherein the actuation pattern is indicative of suggested movement of the ultrasound probe relative to an X-axis, a Y-axis, and a Z-axis to position the ultrasound probe in the desired position.

15. The wearable device of claim 9, wherein the actuation pattern is indicative of suggested movement of the ultrasound probe:
relative to an elevation angle and an azimuth angle to orient a patient-facing surface of the ultrasound probe relative to the patient; or
circumferentially about an axial axis of the ultrasound probe.

16. A method, comprising:
determining, via a processor, a desired imaging position of the ultrasound probe based at least in part on the region of interest and the desired scan plane, wherein the desired imaging position facilitates an acquisition of an ultrasound image of the region of interest, the desired scan plane, or both;
determining, via the processor, a current position of the ultrasound probe relative to the region of interest, the desired scan plane, or both;
determining, via the processor, whether the current position of the ultrasound probe corresponds to the desired imaging position of the ultrasound probe; and
in response to determining that the current position of the ultrasound probe does not correspond to the desired imaging position of the ultrasound probe, transmitting, via the processor, a control signal to a plurality of haptic actuators disposed about a haptic feedback device configured to be worn by an operator of the ultrasound probe, wherein the control signal causes the plurality of haptic actuators to pulse in an actuation pattern indicative of a suggested movement of the ultrasound probe to position the ultrasound probe in the desired imaging position.

17. The method of claim 16, comprising determining, via the processor, a current orientation of the ultrasound probe and a current orientation of the haptic feedback device, and determining, via the processor, a grip style used by the operator of the ultrasound probe based on the current orientation of the ultrasound probe and the current orientation of the haptic feedback device.

18. The method of claim 17, comprising determining, via the processor, the actuation pattern based on the grip style used by the operator of the ultrasound probe, and wherein transmitting the control signal to the plurality of haptic actuators is based on the determined actuation pattern.

19. The method of claim 16, comprising transmitting an additional control signal to the plurality of haptic actuators in response to determining that the current position of the ultrasound probe corresponds to the desired imaging position of the ultrasound probe, and wherein the additional control signal causes the plurality of haptic actuators to pulse in an additional actuation pattern indicative of the determination that the current position corresponds to the desired position.

20. The method of claim 16, wherein each haptic actuator of the plurality of haptic actuators comprise a vibration actuator, the actuation pattern comprises a plurality of actuation patterns indicative of respective suggested movements of the ultrasound probe in six degrees-of-freedom, and the plurality of actuation patterns modulate in intensity, speed, or both, as the ultrasound probe is moved a distance from the desired imaging position.
